(19) United States
(12) Patent Application Publication
(10) Pub. No.: US 2022/0177408 A1
(43) Pub. Date: Jun. 9, 2022

(54) LUBRICATING BASE OIL SYNTHESIZED FROM SUGAR ALCOHOL ESTERS

(71) Applicant: Arkema France, Colombes (FR)

(72) Inventors: Stéphanie LEBRUN, SERQUIGNY (FR); Frédéric MANDON, COLOMBES CEDEX (FR); Guillaume LE, SERQUIGNY (FR)

(73) Assignee: Arkema France, Colombes (FR)

(21) Appl. No.: 17/425,833

(22) PCT Filed: Jan. 29, 2020
(86) PCT No.: PCT/FR2020/050139

§ 371 Date: Jul. 26, 2021

(30) Foreign Application Priority Data
Jan. 29, 2019 (FR) 1900787

(51) Int. Cl. C07C 69/33; C10M 105/40; C07C 67/08

(52) U.S. Cl. CPC C07C 69/33; C10M 2207/2835; C07C 67/08; C10M 105/40

(57) ABSTRACT

The present invention relates to esters of at least one sugar polyol and of at least one C₆-C₁₁ linear fatty acid wherein the sugar polyol is erythritol.

### FIELD OF THE INVENTION

The present invention relates to esters formed from sugar alcohol, in particular sugar polyol, and the use thereof as a lubricant base and also the process for the manufacture thereof.

### TECHNICAL BACKGROUND

Currently, the lubricant base market is dominated by mineral oils of petroleum origin. In 2008, the European production of lubricants amounted to 4.5 million tonnes per year. These lubricant bases are used in various industries as engine oil, cutting oil for chainsaw chains, oil for offshore oil drilling, hydraulic oil for heavy duty equipment and agricultural machinery, etc.

These mineral oils, once used, are not always recycled and cause environmental pollution due to discharge onto the ground, into sewers, into lakes and rivers. In view of the potential impact of these lubricating oils on the environment, the development of ecological and biodegradable lubricant bases is essential, in particular for applications in which the lubricant is liable to escape into the environment.

Vegetable and animal oils, known for several years for their use as a lubricant, could meet this concern for environmental protection because they have the advantage of being environmentally friendly. However, these oils have low thermal stability and also low oxidation resistance compared to mineral oils and are liable to hydrolyze in the presence of water.

Polyol esters, formed from fatty acids attached to an alcohol, have good oxidation stability, good hydrolytic stability, relatively high biodegradability and good low-temperature performance. Biodegradable lubricant compositions of polyol esters derived from palm oil comprising polyols such as neopentyl glycol or trimethylolpropane and products derived from palm oil are described in patent application EP 1 533 360. However, such compositions are only suitable for temperatures ranging from 15° C. to 40° C.

In this context, it therefore remains necessary to develop alternative polyol esters, the structure of which may derive entirely from ingredients of renewable origins, having excellent lubricating properties and also being harmless to humans and the environment.

### SUMMARY OF THE INVENTION

In the context of the present invention, it has been observed that esters of sugar alcohol, in particular of sugar polyol, and of C₆-C₁₁ linear fatty acid have excellent properties for application in lubricants.

The present invention results from the unexpected demonstration, by the inventors, that esters of sugar alcohol, in particular of sugar polyol, notably of erythritol, and of C₆-C₁₁ linear fatty acid have excellent properties for application in lubricants.

Thus, the present invention relates to esters of at least one sugar alcohol, in particular a sugar polyol, and of at least one C₆-C₁₁ linear fatty acid wherein the sugar alcohol, in particular the sugar polyol, is erythritol.

The present invention also relates to the use of an ester of at least one sugar alcohol, in particular a sugar polyol, and of at least one C₆-C₁₁ linear fatty acid as defined above as a lubricant base.

The present invention also relates to a lubricant base composition comprising an ester of at least one sugar alcohol, in particular a sugar polyol, and of at least one C₆-C₁₁ linear fatty acid as defined above.

The present invention also relates to a process for preparing an ester comprising the esterification reaction of at least one C₆-C₁₁ linear fatty acid with at least one sugar alcohol, in particular a sugar polyol, preferably, the process comprises a step of removing the excess acids, and in the absence of at least one of the following steps:
downstream treatment by addition of additive;
addition of catalyst;
addition of organic solvent.

The present invention also relates to esters of at least one sugar alcohol, in particular a sugar polyol, and of at least one C₆-C₁₁ linear fatty acid obtained by the process as defined above.

### DETAILED DESCRIPTION OF THE INVENTION

The lubricant base compositions according to the invention synthesized from esters of at least one polyol and of a fatty acid of renewable origin, such as for example erythritol and n-heptanoic acid (e.g. Oleris® C7 from Arkema) without addition of catalyst and without downstream treatment by addition of additive make it possible to achieve properties in terms of thermal stability which are higher than the usual esters in which the alcohol is not biobased, such as, for example, trimethylolpropane, as is described in detail in the examples below.

Thus, the present invention provides a lubricant base composition of renewable origin which has good oxidation stability, good thermal stability and very good lubricating properties.

Furthermore, since the composition has good flow at low temperature, it is particularly suitable for use at low temperatures, namely typically equal to or less than 0° C.

The term “biodegradable” is used here to denote a compound formed of molecules which can be converted into smaller, less polluting molecules, for example by living microorganisms in the natural environment, such as bacteria, fungi and algae. The end result of this degradation is generally composed of water, carbon dioxide or methane.

Materials or compounds or ingredients that are “derived from renewable resources” or “biobased” is understood to mean renewable natural materials or compounds or ingredients, the stock of which can be reconstituted over a short period on the human timescale. These are in particular raw materials of animal origin or of plant origin. The term “raw materials of renewable origin” or “biobased raw materials” means materials which comprise biobased carbon or carbon of renewable origin. Specifically, unlike materials derived from fossil materials, materials composed of renewable raw materials contain carbon 14 (¹⁴C). The “content of carbon of renewable origin” or content of biobased carbon” is determined by application of the standards ASTM D 6866 (ASTM D 6866-06) and ASTM D 7026 (ASTM D 7026-04).

The viscosity of a fluid means the resistance that it puts up against the internal sliding of its molecules while it is flowing. The viscosity is given for a reference temperature. The kinematic viscosity, expressed in m/s², is calculated using the following formula: υ=η/ρ, where

η is the dynamic viscosity in Pa·s; and
ρ is the density of the fluid in kg/m³.
The kinematic viscosity is also expressed in stokes (St) or in centistokes (cSt).
The kinematic viscosity is measured according to the standard ISO 3104.

Oxidation stability can be determined via two measurements: oxygen induction time and oxygen induction temperature. The oxygen induction time and the oxygen induction temperature can be measured in a differential scanning calorimeter (DSC) according to the standard ISO 11357-6:2018.

The pour point of a product is the minimum temperature at which the product still flows. The pour point is measured according to the standard ISO 3016.

The viscosity index (VI) (unitless) indicates the rate of change in the viscosity of an oil over a given temperature range, usually between 40° C. and 100° C. The viscosity index can be defined as the kinematic viscosity gradient of a material, between 40° C. and 100° C. When the viscosity index is low (less than 100) the fluid displays a relatively large variation in viscosity with temperature. When the viscosity index is high (greater than 150), the fluid has relatively little change in viscosity with temperature. In a variety of applications, a high or very high viscosity index is preferable. The viscosity index is measured according to the test method described in the standard ASTM D 2270.

### Esters

An alcohol is understood to mean a molecule having at least one hydroxyl (—OH) group. A polyol is understood to mean a molecule having at least two hydroxyl (—OH) groups.

Preferably, the polyol according to the invention is an organic compound containing several hydroxyl groups. Preferably, the polyols do not refer to compounds which contain functional groups other than hydroxyl groups. More preferably, the polyol according to the invention is a compound corresponding to the general chemical formula CₙH₂ₙ₊₂Oₙ and having at least two hydroxyl groups.

The esters according to the present invention are formed from at least one sugar alcohol, in particular a sugar polyol, and at least one C₆-C₁₁ fatty acid.

According to one embodiment, the esters according to the present invention may be monoesters, diesters, triesters and tetraesters.

The sugar alcohol, in particular the sugar polyol, according to the invention is preferably obtained from renewable resources. The sugar alcohol, in particular the sugar polyol, according to the invention is preferably biodegradable.

Preferably, the sugar alcohol, in particular the sugar polyol, according to the invention is selected from the group consisting of monosaccharides, disaccharides, trisaccharides, and mixtures thereof.

Preferably, the monosaccharide according to the invention is selected from the group consisting of erythritol, xylose, arabinose, ribose, sorbitol, sorbitan, glucose, sorbose, fructose, xylitol, and mixtures thereof, more preferably from the group consisting of xylose, arabinose, ribose, glucose, sorbose, fructose, and mixtures thereof.

Preferably, the disaccharide according to the invention is selected from the group consisting of maltose, lactose, sucrose, and mixtures thereof.

The trisaccharide according to the invention is preferably selected from the group consisting of raffinose, maltotriose, hydrogenated starch hydrolysates, and mixtures thereof.

More preferably, the sugar alcohol, in particular the sugar polyol, according to the invention is erythritol.

According to one embodiment, the sugar polyol according to the invention is obtained by hydrogenation of a sugar.

The fatty acid according to the invention is preferably derived from renewable resources. The fatty acid according to the invention is preferably linear or branched, saturated or unsaturated, of plant or animal origin.

The fatty acid according to the invention is preferably obtained by trituration of seeds, stones or fruits of plants, in particular oleaginous plants, such as linseed oil, rapeseed oil, sunflower oil, soybean oil, olive oil, palm oil, castor oil, wood oil, corn oil, pumpkin oil, grapeseed oil, jojoba oil, sesame oil, walnut oil, hazelnut oil, almond oil, shea butter, macadamia oil, cottonseed oil, alfalfa oil, rye oil, safflower oil, peanut oil, copra oil, tall oil and argan oil or from animal fats such as tallow fat.

The fatty acid according to the invention is preferably selected from the group consisting of the fatty acids of castor oil, coconut oil, cottonseed oil, dehydrated castor oil, soybean oil, tall oil, rapeseed oil, sunflower oil, linseed oil, palm oil, tung oil, oiticica oil, safflower oil, olive oil, wood oil, corn oil, pumpkin oil, grapeseed oil, jojoba oil, sesame oil, walnut oil, hazelnut oil, almond oil, shea butter, macadamia oil, alfalfa oil, rye oil, peanut oil, copra oil, and argan oil.

The fatty acid according to the invention comprises from 6 to 11 carbon atoms.

Preferably, the fatty acid according to the invention is selected from the group consisting of caproic acid, heptanoic acid, caprylic acid, pelargonic acid, capric acid, furandicarboxylic acid, tetra hydrofuran-2,5-dicarboxylic acid, tetrahydrofuran-3,5-dicarboxylic acid, azelaic acid, decanedioic acid, 10-undecylenic acid, undecanedioic acid, and dodecanedioic acid.

Preferably, the fatty acid according to the invention is a linear fatty acid.

Preferably, linear fatty acids make it possible to increase the viscosity index of the lubricant bases synthesized, to improve the thermal stability thereof and are more readily biodegradable than branched acids, mainly derived from the petroleum industry.

Preferably, the fatty acid according to the invention is derived from castor oil.

The fatty acid derived from castor oil is understood to mean the fatty acid present in the oil and/or the fatty acids which can be obtained at the end of a chemical conversion. For example, heptanoic acid and/or 10-undecylenic acid can be obtained from castor oil, typically, by a step of thermal cracking of methyl ricinoleate which originates from the transesterification of castor oil. The fatty acid according to the invention is preferably n-heptanoic acid. Preferably also, the fatty acid according to the invention is the n-heptanoic acid Oleris® (Arkema).

Preferably, the n-heptanoic acid is derived from castor oil.

Preferably, the ester according to the invention is formed from a sugar alcohol, in particular from a sugar polyol, according to the invention, of which at least 3 alcohol groups, preferably 4 alcohol groups, are esterified with fatty acids according to the invention.

Preferably also, the weight ratio of the fatty acid according to the invention to the sugar alcohol, in particular the sugar polyol, according to the invention is within the range of from 4:1 to 10:1. More preferably, the weight ratio of the fatty acid according to the invention to the sugar alcohol, in particular the sugar polyol, according to the invention is around 5:1.

Preferably, a fraction of at least 50% by weight, preferably 75% by weight of the ester is derived from renewable resources relative to the total weight of the ester.

Preferably, the ester according to the invention has an oxygen induction time measured in a differential scanning calorimeter at 150° C. of more than 2 hours.

Preferably, the ester according to the invention has an oxygen induction temperature measured in a differential scanning calorimeter of greater than 200° C.

The ester according to the invention preferably comprises a pour point below −30° C., preferably between −50° C. and −30° C., more preferably of around −42° C.

The ester according to the invention preferably comprises a kinematic viscosity of between 14 and 30 mm²/s at 40° C., and/or less than 4.5 mm²/s at 100° C., which are measured according to the standard ISO 3104.

### Process

The process for preparing the esters according to the invention from sugar alcohol, in particular sugar polyol, and fatty acid according to the invention can be carried out according to the usual esterification techniques well known to those skilled in the art.

Preferably, the esterification process according to the invention comprises a step of esterifying at least one sugar alcohol according to the invention, in particular a sugar polyol, in the presence of at least one C₆-C₁₁ linear fatty acid according to the invention in excess, with or without a catalyst.

The esterification step according to the invention is preferably carried out at a temperature between 140° C. and 250° C. for a period of from 0.5 to 12 hours, preferably from 1 to 10 hours, more preferably from 2 to 9 hours.

The esterification step according to the invention is preferably carried out under an inert atmosphere.

Preferably, the process for preparing the esters according to the invention is carried out under controlled vacuum so as to remove the excess acid. The esterification process according to the invention may comprise a step of adding an absorbent such as alumina, silica gel, zeolites, activated carbon, and clay.

The process according to the invention may further comprise a step of adding water and base to simultaneously neutralize the residual organic and mineral acids and/or hydrolyze the catalyst. In this case, the process according to the invention may comprise a step of removing the water used by heating and placing under vacuum.

The process according to the invention may also comprise a step of filtering the solids of the ester mixture containing most of the excess acid mixture used in the esterification reaction.

The process according to the invention may comprise a step of removing the excess acids by steam extraction or by any other method of distillation and recycling of the acid in the reaction vessel.

According to one embodiment, the process of the invention comprises a step of removing excess acids, preferably carried out by vacuum distillation.

Preferably, the compound obtained by the process according to the invention is purified by reduced-pressure distillation of the unreacted acid. The distillation is preferably carried out under vacuum for 15 minutes to 2 hours. The distillation is further preferably carried out at a temperature of between 140° C. and 180° C. The amount of free acid remaining after the distillation step can be reduced by treatment with epoxy esters, by neutralization with any suitable alkaline material such as lime, alkali metal hydroxides, alkali metal carbonates or basic alumina. When a treatment with epoxy esters is carried out, a second reduced-pressure distillation may be carried out to remove the excess epoxy ester. When an alkaline treatment is carried out, washing with water may be carried out to remove excess unreacted alkaline material.

The method according to the invention may comprise a step of removing any residual solid material from the ester extracted during a final filtration.

Preferably, the fatty acid according to the invention is present in the reaction to form the ester according to the invention in an excess of around 10 to 50 mol %, preferably 10 to 30 mol %, relative to the amount of sugar alcohol, in particular sugar polyol, used.

The process according to the invention can be carried out in the presence of a catalyst. The catalyst can be any catalyst well known to those skilled in the art for esterification reactions. Preferably, the catalyst is selected from the group consisting of tin chloride, sulfuric acid, p-toluenesulfonic acid, methanesulfonic acid, sulfosuccinic acid, hydrochloric acid, phosphoric acid, catalysts based on zinc, copper, tin, titanium, zirconium or tungsten; alkali metal salts such as sodium or potassium hydroxide, sodium or potassium carbonate, sodium or potassium ethoxide, sodium or potassium methoxide, zeolites and acidic ion exchangers, or mixtures thereof.

Preferably, no downstream treatment step by addition of an additive is carried out during the process for preparing the ester according to the invention.

The term “downstream treatment by addition of an additive” is understood to mean one or more of the steps typically carried out at the end of the esterification step, as described above, namely, the step of adding an absorbent, the step of adding water and base, the step of filtering solids from the mixture of esters and/or the step of removing the excess acids.

Preferably, the process for preparing the ester is carried out without a catalyst.

Preferably, the process for preparing the ester is carried out without addition of organic solvent.

Preferably, the process for preparing the ester is carried out in the absence of at least one, preferably at least two, more preferably all of the following steps:
downstream treatment by addition of additive;
addition of catalyst;
addition of organic solvent.

Preferably, the reaction is carried out for a sufficient time to obtain a content of tetraesters greater than or equal to 80% by weight relative to the total amount of ester. More preferably, the reaction is carried out for a sufficient time to obtain a content of tetraesters greater than or equal to 93% by weight relative to the total amount of ester.

### Use

The esters according to the invention are preferably used as they are as lubricant base or lubricant base oil.

The esters according to the invention may also be used as a mixture with other base oils, such as mineral oils, highly refined mineral oils, polyalphaolefins (PAO), polyalkylene glycols (PAG), phosphate esters, silicone oils, diesters, polyisobutylenes and polyol esters.

In particular, the esters according to the invention are useful for the preparation of a lubricant base composition. The lubricant base composition according to the invention can be used in all types of industries, in particular as automotive lubricants, as metalworking oils, as hydraulic oils, as turbine oils, or else as aircraft oils.

Preferably, the composition according to the invention may comprise a content of tetraesters greater than or equal to 80% by weight relative to the total amount of ester. More preferably, the composition may comprise a content of tetraesters greater than or equal to 93% by weight relative to the total amount of ester.

The composition according to the invention may comprise, in addition to the esters according to the invention, one or more additives. Preferably, the additives are selected from the group consisting of antioxidants, thermal stability improvers, corrosion inhibitors, metal deactivators, lubricant additives, viscosity index improvers, pour point depressants, detergents, dispersants, antifoaming agents, antiwear agents, and extreme pressure additives.

Preferably, the amount of additives in the composition according to the invention does not exceed 10% by weight, preferably 8% by weight, more preferably 5% by weight relative to the total weight of the lubricant base composition.

Preferably, the amount of antioxidants used is between 0.01% and 5% relative to the total weight of the lubricant base composition.

Preferably, the amount of corrosion inhibitors is between 0.01% and 5% by weight relative to the total weight of the lubricant base composition.

Preferably, the amount of metal deactivators is between 0.001% and 0.5% by weight relative to the total weight of the lubricant base composition.

Preferably, the amount of lubricant additives is between 0.5% and 5% by weight relative to the total weight of the lubricant base composition.

Preferably, the amount of viscosity index improvers is between 0.01% and 2% by weight relative to the total weight of the lubricant base composition.

Preferably, the amount of pour point depressants is between 0.01% and 2% by weight relative to the total weight of the lubricant base composition.

Preferably, the amount of detergents is between 0.1% and 5% by weight relative to the total weight of the lubricant base composition.

Preferably, the amount of dispersants is between 0.1% and 5% by weight relative to the total weight of the lubricant base composition.

Preferably, the amount of antifoaming agents is between 0.01% and 2% by weight relative to the total weight of the lubricant base composition.

Preferably, the amount of antiwear agents is between 0.01% and 2% by weight relative to the total weight of the lubricant base composition.

Preferably, the amount of extreme pressure additives is between 0.1% and 2% by weight relative to the total weight of the lubricant base composition.

Antioxidants and thermal stability improvers can be chosen from any antioxidants and thermal stability improvers well known to those skilled in the art. By way of example, the antioxidant and the thermal stability improver can be selected from the group consisting of:
diphenylamine, dinaphthylamine, phenylnaphthylamine, in which the phenyl group or the naphthyl group can be substituted, for example by N,N′-diphenylphenylenediamine, p-octyldiphenylamine, p,p-dioctyldiphenylamine, N-phenylnaphthylamine, N-phenyl-2-naphthylamine, N-(p-dodecyl)phenyl-2-naphthylamine, di-1-naphthylamine, and di-2-naphthylamine groups;
phenothazines, such as N-alkylphenothiazines;
imino(bisbenzyl); and
hindered phenols such as 6-(t-butyl)phenol, 2,6-di-(t-butyl)phenol, 4-methyl-2,6-di-(t-butyl) phenol, 4,4′-methylenebis(2,6-di-(t-butyl)phenol).

The metal deactivators can be chosen from any metal deactivators well known to those skilled in the art. By way of example, the metal deactivators can be selected from the group consisting of imidazole, benzamidazole, 2-mercaptobenzthiazole, 2,5-dimerca ptothiadiazole, salicylidene-propylenedia mine, pyrazole, benzotriazole, tolutriazole, 2-methylbenzamidazole, 3,5-dimethylpyrazole, and methylenebis(benzotriazole). Other examples of metal deactivators or corrosion inhibitors include:
organic acids and esters, metal salts and anhydrides thereof, such as N-oleylsarcosine, sorbitan monooleate, lead naphthenate, dodecenylsuccinic acid and the partial esters and amides thereof, and 4-nonylphenoxyacetic acid;
aliphatic and cycloaliphatic primary, secondary and tertiary amines and amine salts of organic and inorganic acids, such as oil-soluble alkylammonium carboxylates;
heterocyclic compounds containing nitrogen, such as thiadiazoles, substituted imidazolines, and oxazolines;
quinolines, quinones and anthraquinones;
propyl gallate;
barium dinonylnaphthalenesulfonate;
derivatives of esters and amides of alkenylsuccinic anhydrides or acids, dithiocarbamates, dithiophosphates;
amine salts of alkyl acid phosphates and derivatives thereof.

The lubricant additives can be chosen from any lubricant additives well known to those skilled in the art. Mention may be made, as examples of lubricant additives, of long-chain derivatives of fatty acids and of natural oils, such as esters, amines, amides, imidazolines and borates.

The viscosity index improvers can be chosen from any viscosity index improvers well known to those skilled in the art. Mention may be made, as examples of viscosity improvers, of polymethacrylates, copolymers of vinylpyrrolidone and of methacrylates, polybutenes and styrene-acrylate copolymers.

The pour point depressants can be chosen from any pour point depressants well known to those skilled in the art. Mention may be made, as examples of pour point depressants, of polymethacrylates such as methacrylate-ethylene-vinyl acetate terpolymers; alkylated naphthalene derivatives; and the products of urea-catalyzed Friedel-Crafts condensation with naphthalene or phenols.

The detergents and dispersants can be chosen from any detergents and dispersants well known to those skilled in the art. Mention may be made, as examples of detergents and dispersants, of polybutenylsuccinic acid amides; polybutenylphosphonic acid derivatives; aromatic sulfonic acids substituted by a long-chain alkyl and the salts thereof; and metal salts of alkyl sulfides, alkylphenols and condensation products of alkylphenols and aldehydes.

The anti-foaming agents can be selected from any anti-foaming agents well known to those skilled in the art. Mention may be made, as examples of anti-foaming agents, of polymers of silicone and certain acrylates.

The antiwear agents and extreme pressure additives can be chosen from any antiwear agents and extreme pressure additives. Mention may be made, as examples of antiwear agents and extreme pressure additives, of:
sulfurized fatty acids and fatty acid esters, such as sulfurized octyl tallate;
sulfurized terpenes;
sulfurized olefins;
organopolysulfides;
organophosphorus derivatives comprising amine phosphates, alkyl acid phosphates, dialkyl phosphates, aminedithiophosphates, trialkyl and triaryl phosphorothionates, trialkyl and triaryl phosphines, and dialkyl phosphites such as amine salts of phosphoric acid monohexyl ester, amine salts of dinonylnaphthalenesulfonate, triphenyl phosphate, trinaphthyl phosphate, diphenyl cresyl phosphate, and phenylphenyl phosphates, naphthyl diphenyl phosphate, triphenylphosphorothionate;
dithiocarbamates, such as an antimony dialkyldithiocarbamate;
chlorinated and/or fluorinated hydrocarbons and xanthates.
   The invention will be further explained with the aid of the following nonlimiting examples.

### EXAMPLES

The inventors have studied the properties of an ester according to the present invention for application in lubricants.

### 1. Preparation of the Ester

### 2 Samples are Prepared:

ester of erythritol and of n-heptanoic acid (ester according to the invention); and
ester of trimethylolpropane and of n-heptanoic acid (comparative example 1).
   Synthesis of an Ester of Erythritol and of n-Heptanoic Acid (Ester According to the Invention):

Erythritol (14.7 g, 0.12 mol) and n-heptanoic acid (81.7 g, 0.62 mol) are loaded into a 250 ml three-necked flask equipped with a stirrer, a thermometer, a condenser and a nitrogen inlet. The reaction mixture was heated at 210° C. under a nitrogen atmosphere for a period of 7 h 30 min, until the theoretical amount of water was collected. The crude product is then distilled at a temperature of 180° C. and under maximum vacuum for 1 hour 30 min to remove the excess n-heptanoic acid in order to obtain 66.5 g of product with an acid number of 0.1 mgKOH/g.

The kinematic viscosities, the viscosity index (VI) and the pour point of the product are evaluated and reported in table no. 2.

The chemical composition of the product was established by gas chromatography as follows: 94.1% of tetraester of erythritol and n-heptanoic acid, 2.2% of triester of erythritol and n-heptanoic acid and 2.9% anhydroester of erythritol and n-heptanoic acid.

### Synthesis of an Ester of Trimethylolpropane and n-Heptanoic Acid (Comparative Example 1)

Trimethylolpropane (53.8 g, 0.4 mol) and n-heptanoic acid (181.5 g, 1.38 mol) are loaded into a 500 ml three-necked flask equipped with a stirrer, a thermometer, a condenser and a nitrogen inlet. The reaction mixture was heated at 185° C. under a nitrogen atmosphere for a period of 3 h, until the theoretical amount of water was collected. Zirconium tetrabutanolate (1.5 g, at 80% in butanol, 0.5% by weight/total weight of the reactants) is then added batchwise to the reactor. The assembly is gradually placed under maximum vacuum at 150° C. for 3 hours 30 minutes to distil off the excess unreacted acid and results in 187.4 g of product. A downstream treatment with activated basic alumina is carried out on the crude reaction product and results in an oil with an acid number of 0.1 mgKOH/g.

The kinematic viscosities, the viscosity index (VI) and the pour point of the product are evaluated and reported in table no. 2.

The chemical composition of the product was established by gas chromatography as follows: 98.8% trimethylolpropane triheptanoate and 0.03% trimethylolpropane diheptanoate.

### 2. Measurement of the Oxidation Resistance

Oxidation stability is determined via two measurements: oxygen induction time and oxygen induction temperature. Oxygen induction time and oxygen induction temperature are measured in a differential scanning calorimeter (DSC).

For the measurement of the oxygen induction time, the sample is heated to 150° C. and then maintained at constant temperature. It is then exposed to an oxidizing atmosphere. The time between contact with oxygen and the onset of oxidation is the oxygen induction time.

For the measurement of the oxygen induction temperature, the sample is heated with a constant heating rate under an oxidizing atmosphere until the reaction begins. The oxygen induction temperature is the temperature at which the oxidation reaction begins.

The results are presented in Table 1 below:

TABLE 1
oxidation resistance measurement

| | Ester of erythritol | |
|---|---|---|
| | and n- | Ester of |
| | heptanoic acid: | trimethylolpropane |
| | Ester according | and n-heptanoic acid: |
| Product | to the invention | Comparative example 1 |
| Oxygen induction time at | >2 hours | >2 hours |
| 150° C. | | |
| Oxygen induction | 202 | 196 |
| temperature (° C.) | | |

The measurements show that the oxygen induction times at 150° C. of the two samples are similar. The ester according to the invention has a higher oxygen induction temperature than that of the comparative example. Consequently, the ester according to the invention has better oxidation resistance properties than a usual ester synthesized from a non-biobased alcohol.

### 4. Measurement of the Kinematic Viscosity

The kinematic viscosity was measured at 40° C. and at 100° C. according to the standard ISO 3104.

The results, expressed in mm²/s, are presented in table 2 below.

### 5. Measurement of the Viscosity Index

The viscosity index (unitless) is measured according to the test method described in standard ASTM D 2270. The results are presented in table 2 below:

### 6. Measurement of the Pour Point

The pour point, expressed in ° C., is measured according to the standard ISO 3016. The results are presented in table 2 below:

TABLE 2
measurement of the kinematic viscosity, viscosity index and pour point.

| | Ester of erythritol | Ester of |
|---|---|---|
| | and n- | trimethylolpropane |
| | heptanoic acid: | and n- |
| | Ester according | heptanoic acid: |
| | to the | Comparative |
| Product | invention | example 1 |
| Kinematic viscosity at 40° C. | 17.4 | 14.0 |
| (mm²/s) ISO 3104 | | |
| Kinematic viscosity at | 3.9 | 3.4 |
| 100° C. (mm²/s) ISO 3104 | | |
| Viscosity index (VI) ASTM | 127 | 118 |
| D2270 | | |
| Pour point (° C.) ISO 3016 | −42 | −63 |

These results show that the ester according to the invention synthesized solely from substances of renewable origin without addition of catalyst and without downstream treatment by addition of additive, unlike the comparative example, has kinematic viscosities at 40° C. and 100° C. close to those of the comparative example. The ester according to the invention displays a higher viscosity index, which means that the lubricant base according to the invention has a more stable viscosity as a function of the temperature.

The lubricant base of the invention displays a higher pour point, correlated with the higher melting point of erythritol (120° C.) than that of trimethylolpropane (60° C.) of the comparative example, but this value remains relatively low and advantageous for application in lubricants.

1. Esters of at least one sugar polyol and of at least one C₆-C₁₁ linear fatty acid, wherein the sugar polyol is erythritol.

2. The esters as claimed in claim 1, wherein the C₆-C₁₁ linear fatty acid is n-heptanoic acid.

3. The esters as claimed in claim 1, wherein the weight ratio of the C₆-C₁₁ linear fatty acid to the sugar polyol is at least 5:1.

4. The esters as claimed in claim 1, wherein the C₆-C₁₁ linear fatty acid is derived from renewable resources.

5. The esters as claimed in claim 1, wherein the C₆-C₁₁ linear fatty acid is derived from castor oil.

6. The use of an ester of at least one sugar polyol and of at least one C₆-C₁₁ linear fatty acid as defined in claim 1 as a lubricant base.

7. A composition of a lubricant base comprising an ester of at least one sugar polyol and of at least one C₆-C₁₁ linear fatty acid as defined in claim 1.

8. A process for preparing an ester comprising a step of esterifying at least one sugar polyol in the presence of at least one C₆-C₁₁ linear fatty acid in excess.

9. The process as claimed in claim 8, wherein the process comprises a step of removing excess acids.

10. The process as claimed in claim 8, wherein the process is carried out in the absence of at least one of the following steps:
downstream treatment by addition of additive;
addition of catalyst;
addition of organic solvent.

11. The process as claimed in claim 8, wherein the reaction is carried out for a sufficient time to obtain a content of tetraesters greater than or equal to 80% by weight relative to the total amount of ester.

12. The process as claimed in claim 8, wherein the sugar polyol is erythritol.

13. The process as claimed in claim 8, wherein the C₆-C₁₁ linear fatty acid is n-heptanoic acid.

14. The process as claimed in claim 8, wherein the C₆-C₁₁ linear fatty acid is derived from renewable resources.

15. The process as claimed in claim 13, wherein the C₆-C₁₁ linear fatty acid is derived from castor oil.

16. The process as claimed in claim 8, wherein the weight ratio of the C₆-C₁₁ linear fatty acid to the sugar polyol is at least 5:1.

17. Esters of at least one sugar polyol and at least one C₆-C₁₁ linear fatty acid obtained by the process as claimed in claim 8.
